(19) United States
(12) Patent Application Publication
(10) Pub. No.: US 2019/0261679 A1
(43) Pub. Date: Aug. 29, 2019

(54) BOWL ASSEMBLY FOR A VAPOR SMOKING DEVICE

(71) Applicant: Mohammed Alam AlShamma, Tripoli (LB)

(72) Inventor: Mohammed Alam AlShamma, Tripoli (LB)

(21) Appl. No.: 16/288,717

(22) Filed: Feb. 28, 2019

Related U.S. Application Data
(60) Provisional application No. 62/636,288, filed on Feb. 28, 2018.

(51) Int. Cl. A24F 1/30

(52) U.S. Cl. CPC A24F 1/30; A61M 11/041

(57) ABSTRACT

A vaporizing device is provided for a vapor smoking device, includes a removable cartridge defining an internal cavity, and is configured to be disposed on the vapor smoking device. A plurality of porous granules is disposed within the internal cavity. The porous granules have a vaporizing liquid absorbed therein.

### CROSS-REFERENCE TO RELATED APPLICATIONS

This patent application claims priority to U.S. Provisional Patent Application No. 62/636,288, filed on Feb. 28, 2018, the disclosure of which is incorporated by reference herein in its entirety.

### TECHNICAL FIELD

The invention relates to smoking devices, and more specifically, to a bowl assembly with cartridges for vaporizing devices.

### BACKGROUND OF THE INVENTION

There are a variety of devices and systems used for smoking tobacco, including pipes, cigarettes, water pipes and shishas (also referred to as hookahs). In recent years, vaporizing or “vaping” has become more popular, particularly among tobacco smokers. A popular vaporizing device is a so-called e-cigarette. Because vaporizers use a liquid in place of tobacco and other solid substances, devices designed for tobacco use may not be readily or conveniently used for vaporizing.

### SUMMARY

In one aspect, the present invention is directed to a vaporizing device comprising: a removable cartridge defining an internal cavity and configured to be disposed on a vapor smoking device; and a plurality of porous granules disposed within the internal cavity, the plurality of porous granules having a vaporizing liquid absorbed into the plurality of porous granules.

In another aspect, the present invention is directed to a bowl assembly for a vapor smoking device, the bowl assembly comprising: a removable cartridge defining an internal cavity and configured to be disposed on a vapor smoking device; a plurality of porous granules disposed within the internal cavity, the plurality of porous granules having a vaporizing liquid absorbed into the plurality of porous granules; and a bowl portion having a recess configured to receive a lower portion of the removable cartridge.

### BRIEF DESCRIPTION OF THE DRAWINGS

FIG. 1 is a side view of a shisha smoking device according to an embodiment of the present invention;

FIG. 2 is side view of a vaporizing assembly according to one embodiment of the present invention, the vaporizing assembly including a bowl portion and a cartridge, and is configured for use with a smoking device such as the smoking device of FIG. 1;

FIG. 3 is a perspective view of the vaporizing assembly of FIG. 2;

FIG. 4 is a side view of the vaporizing assembly of FIG. 2, showing the bowl portion separated from the cartridge;

FIG. 5 is an upper perspective view of the vaporizing assembly of FIG. 2, showing the bowl portion separated from the cartridge;

FIG. 6 is a lower perspective view of the vaporizing assembly of FIG. 2, showing the bowl portion separated from the cartridge;

FIG. 7 is a cross-sectional view of one embodiment of a cartridge configured to house fluid or porous granules with fluid therein according to one embodiment of the present invention;

FIG. 8 is a cross-sectional view of one embodiment of a bowl portion configured hold the cartridge of FIG. 7;

FIG. 9 is a side view of another embodiment of a cartridge according to the present invention;

FIG. 10 is an upper perspective view of the cartridge of FIG. 9;

FIG. 11 is another upper perspective view of the cartridge of FIG. 9;

FIG. 12 is a lower perspective view of the cartridge of FIG. 9;

FIG. 13 is a perspective view of the cartridge of FIG. 9 in a disassembled configuration;

FIG. 14 is a top view of another embodiment of a cartridge according to the present invention, showing an example of dimensions of the cartridge;

FIG. 15 is a top view of another embodiment of a bowl portion of a vaporizing assembly according to the present invention, showing an example of dimensions of the bowl portion;

FIG. 16 is a cross-sectional view of the bowl portion of FIG. 15; and

FIG. 17 is a cross-sectional view of the cartridge of FIG. 14.

The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention.

### DETAILED DESCRIPTION OF THE PREFERRED EMBODIMENTS

The following embodiments are merely illustrative and are not intended to be limiting. It will be appreciated that various modifications and/or alterations to the embodiments described herein may be made without departing from the disclosure and any modifications and/or alterations are within the scope of the contemplated disclosure.

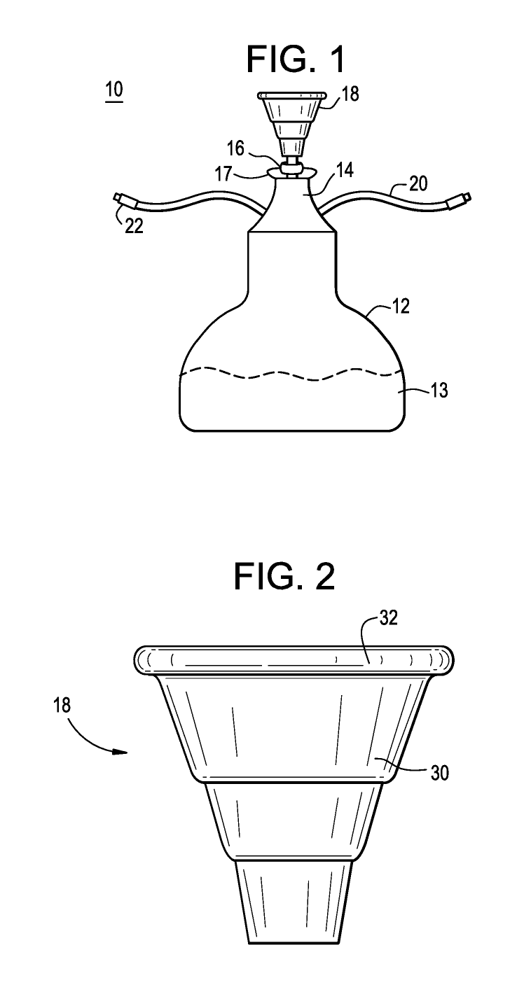

FIG. 1 illustrates one embodiment of a shisha (or hookah) smoking device 10 in accordance with the present invention. The device 10 includes a base 12 into which water or another suitable liquid 13 may optionally be added, a body pipe 14, and an adapter 16 on which a shisha bowl assembly 18 is mounted. The device also includes one or more hoses 20, each of which is connected to a mouthpiece 22.

As discussed further below, the shisha bowl assembly 18 is configured as a vaporizing unit, which may be installed on the top of the smoking device 10. For example, the adapter 16 may include a heating element 17 configured to heat a vaporizing fluid in the shisha bowl assembly 18, or the heating element 17 is configured to be placed on top of the shisha bowl assembly 18 or in proximity thereto. Alternatively, the shisha bowl assembly 18 itself may be equipped with the heating element 17.

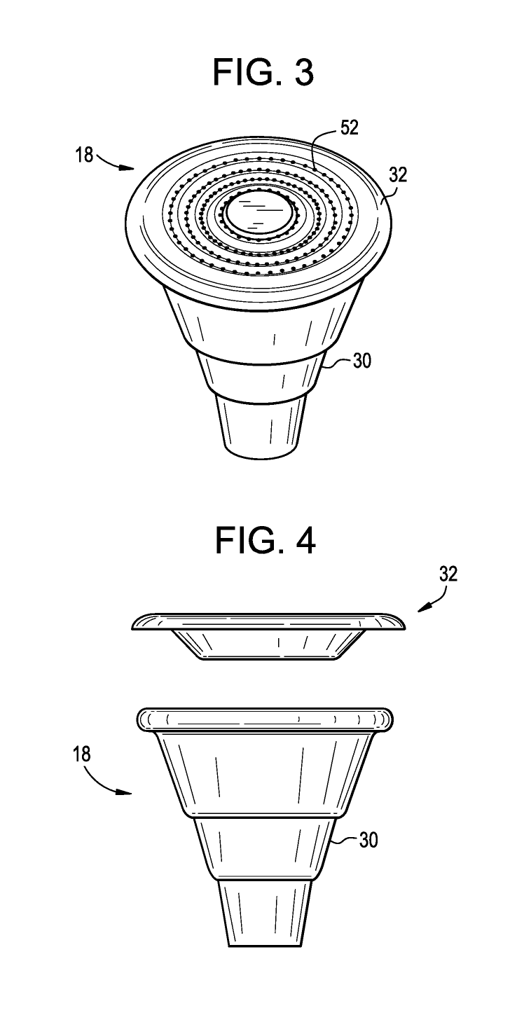

FIGS. 2 and 3 show one embodiment of the shisha bowl assembly 18 as assembled for use in accordance with the present invention. The shisha bowl assembly 18 includes a bowl portion 30 and a removable cartridge 32 mounted on or otherwise operably coupled to the bowl portion 30. The bowl portion 30 may be disposable or non-disposable. For example, in one embodiment, the bowl portion 30 is made from a fire treated clay material with or without a ceramic glaze coat. Other materials that the bowl portion 30 may be made of include, for example, ceramic, plastic, metal, metal alloys and any other suitable material. The cartridge 32 also may be disposable or non-disposable. In one embodiment, the cartridge 32 is fabricated from aluminum. Other materials that the cartridge 32 may be made of include, for example, ceramic, plastic, metal, metal alloys and any other suitable material. In one embodiment, the cartridge 32 is a disposable aluminum cartridge.

FIGS. 4 to 6 show an embodiment of the shisha bowl assembly 18 in which the cartridge 32 is removed from the bowl portion 30. As discussed further below, in one embodiment, the cartridge 32 is self-contained, may be disposable, and houses a vaporizing fluid 33. The cartridge 32, in one embodiment, does not include or employ any adhesive or securing mechanism. Instead, the cartridge 32 can be placed on the bowl portion 30 without a securing mechanism.

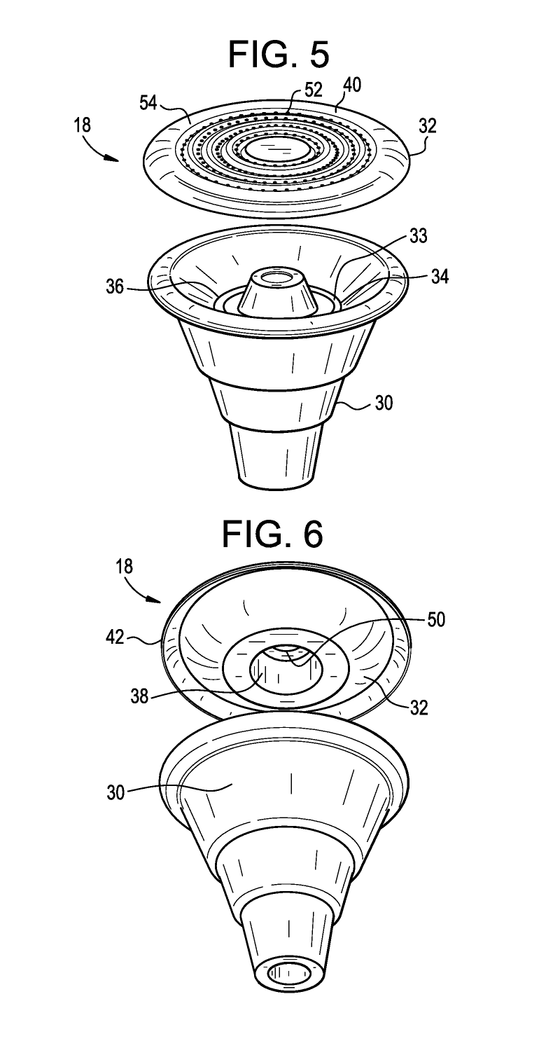

In one embodiment, as shown in FIG. 5, the bowl portion 30 includes a recess 34 having a shape corresponding to a shape of at least a lower component of the cartridge 32. For example, in one embodiment, the recess 34 may be circular about a circumference of the bowl portion 30 as shown or have any other suitable shape. The bowl portion 30 also forms a socket 36 that protrudes upwardly from a base or lower surface of the bowl portion 30 through the recess 34 and is configured to engage a similarly shaped receiving portion 38 extending downwardly from the cartridge 32, as shown in FIG. 6. In use, the cartridge 32 is placed on the bowl portion 30 so that the cartridge 32 rests in the recess 34, and the socket 36 is inserted into the receiving portion 38. The heating element 17 may be engaged to heat a vaporizing fluid disposed in the cartridge 32, for example, by placing the heating element 17 on top of the cartridge 32 or in the adapter 16 (FIG. 1). The heat triggers a phase-change such that the vaporizing fluid 33 evaporates or vaporizes to form a vapor.

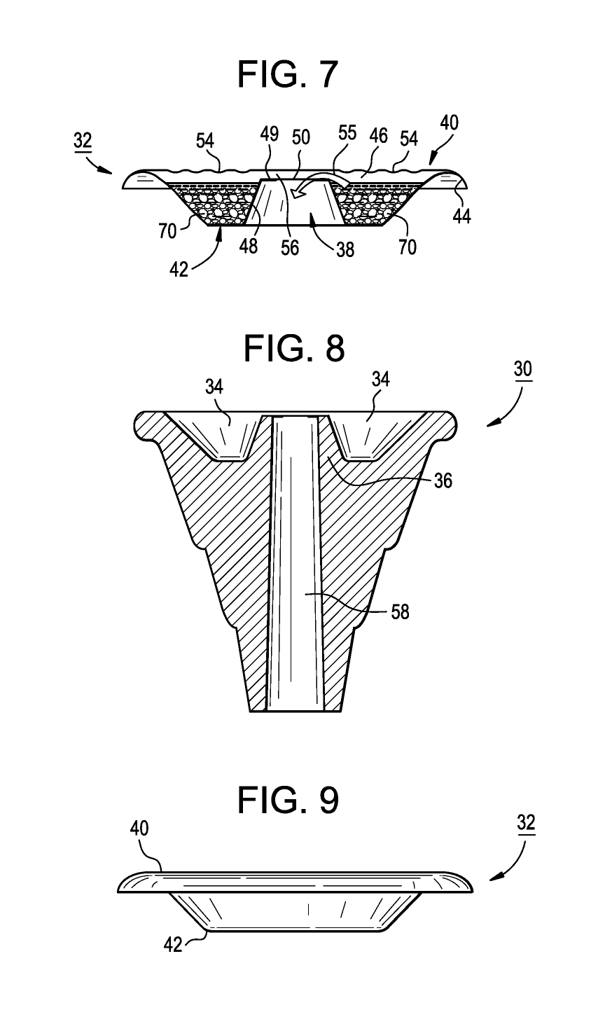

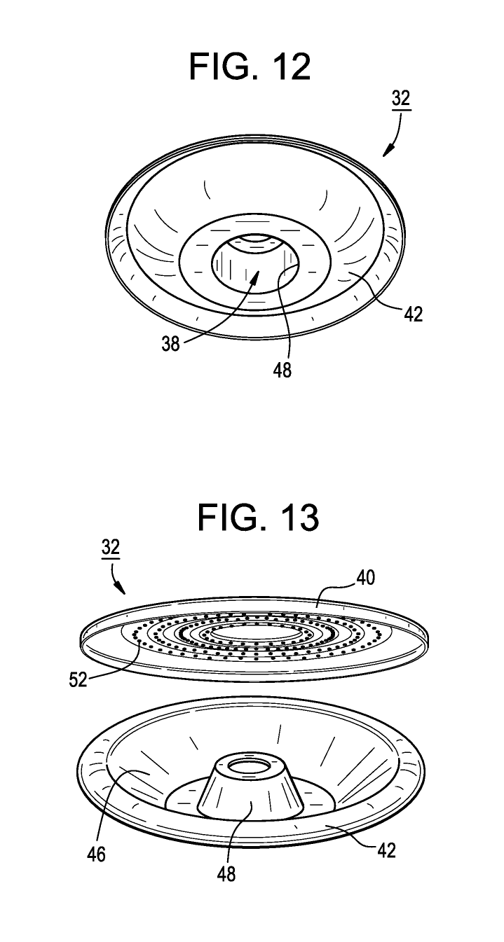

FIGS. 7 to 13 show an embodiment of the cartridge 32 (FIGS. 7 and 9 to 13) and a corresponding bowl portion 30 (FIG. 8). FIG. 7 shows a cross-section of one embodiment of the cartridge 32. In this embodiment, the cartridge 32 includes an upper component 40 and a lower component 42, which may be manufactured as separate parts and sealed together to form an at least fluid tight seal 44 that binds the outer edges of the components together. In one embodiment, the upper component 40 and/or the lower component 42 are comprised of food-grade aluminum. Examples of such food-grade aluminum include alloy 8011-26, alloy 8011-0, or equivalents thereof. In one embodiment, the thickness of the aluminum or other material making up the upper component 40, or lid, and/or the lower component 42, or container, may be, e.g., about 0.4 mm to about 1.6 mm.

The components 40 and 42, when sealed or assembled together, for example by press fit or press mold or the like, define an internal cavity 46 in which the vaporizing fluid 33 is disposed. As noted above, the vaporizing fluid 33 is configured to form a vapor when sufficient heat is applied.

The cartridge 32 may have a number of features that facilitate the vaporizing process. For example, the receiving portion 38 is formed by an upwardly extending feature 48 having a conical shape and terminating at a relatively flat surface 49. A hole 50 is disposed in the flat surface 49. The upwardly extending feature 48, the flat surface 49 and the hole 50 cooperate to permit the vapor formed by heating to escape the cartridge 32 and be drawn through the bowl portion 30.

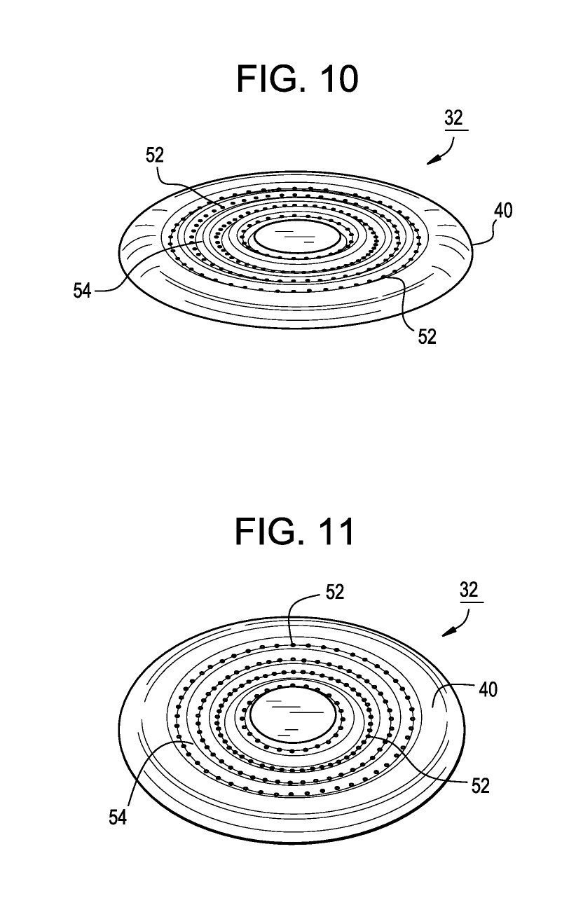

Other features may be included in the cartridge 32 to facilitate heat transfer and distribution, such as for example one or more holes 52 in a surface of the upper component 40 (FIGS. 10 and 11). The holes 52 act to facilitate heat transfer from the heating element 17 to the internal cavity 46. In the embodiments shown in FIGS. 1 to 14, the holes 52 are arranged in concentric circles surrounding a central point of the upper component 40, but the arrangement of the holes 52 is not so limited, as there may be any suitable number of holes 52, which may have a variety of sizes and arrangements. The upper component 40 may also have one or more heat distribution grooves 54 formed by indentations having a selected depth. In one embodiment, such a selected depth is less than or equal to about 0.7 mm. The grooves 54 facilitate distributing heat evenly throughout the cartridge 32. There may be any number of grooves 54 having any selected depth and configuration or arrangement.

In one embodiment, the lower component 42 and the upper component 40 are shaped so that a gap 56 is formed between the relatively flat surface 49 and the upper component 40. As shown in FIG. 7, the gap 56 provides a fluid/vapor flow path 55 from the internal cavity 46 to the hole 50. When the cartridge 32 (e.g., a disposable aluminum cartridge) is installed on the bowl portion 30 (e.g., a clay bowl portion), the gap 56 and the hole 50 are in fluid/vapor communication with a corresponding air duct 58 in the bowl portion 30. In use, the heating element 17 is activated to vaporize some of the vaporizing fluid as a user draws in or inhales on the mouthpiece 22 (see FIG. 1), and the vapor is drawn into the air duct 58 via the gap 56 and the hole 50, and travels though the body pipe 14.

Referring again to FIG. 7, in one embodiment, the cartridge 32 includes a plurality of porous granules 70, which can have any suitable size and/or shape, disposed within the internal cavity 46. For example, the granules 70 may be generally spherical granules, but can have any suitable shape. The porous granules 70 may be made from ceramic or other material configured to be used in high temperature environments, such as one or more types of Zeolite materials, such as Zeolite molecular sieves configured as granules having an approximate diameter of, e.g., about 1.5 mm to about 4.0 mm.

In one embodiment, the porous granules 70 perform the function of absorbing the vaporizing fluid 33. The granules 70 absorb the vaporizing liquid, and can provide an even vaporization process as the vaporizing liquid may be generally evenly distributed through the granules 70. In one example, the granules can absorb approximately 12 ml of liquid, which provides about 400 up to 600 puffs of vapor.

An example of a vaporizing fluid 33 (commonly known as E-liquid) includes glycerin (C₃H₈O₃), which is fit for human consumption and clear of any poisonous or harmful ingredients. The selected purity of the glycerin is, e.g., up to about ninety-nine percent (99%) without any unwanted flavors or aftertaste, or between about seventy to eighty percent (70-80%) (depending on the added flavor). The vaporizing fluid 33 in this example also includes food-grade propylene glycol (C₃H₈O₂). The percentage of the vaporizing fluid 33 made up of propylene glycol may vary, e.g., is greater than about twenty percent (20%).

The vaporizing fluid 33 may include additional optional components such as flavorings and nicotine. For example, various food-grade flavors can be incorporated in the vaporizing fluid 33. The percentage of the vaporizing fluid 33 made from such flavors can be, e.g., about five to fifteen percent (5-15%). The amount of nicotine may vary based on the desired strengths, and is typically about 4 mg or less.

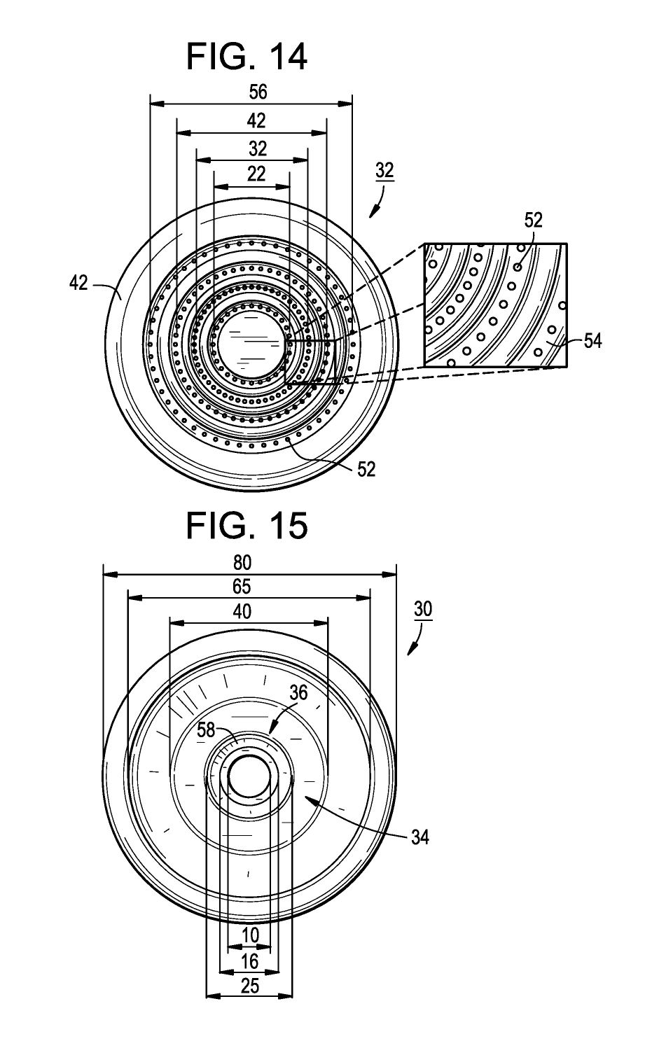

FIGS. 14 to 17 illustrate exemplary dimensions (in millimeters or mm) of the bowl portion 30 and the cartridge 32. It is noted that these dimensions are provided only for illustrative purposes and are not intended to limit the dimensions.

Referring to FIG. 14, the holes 52 are arranged in concentric circles about the center of the upper component 40, each defining a respective diameter. In this example, the circles have diameters of 22 mm, 32 mm, 42 mm and 56 mm. The grooves 54 are formed by indentations having a depth of about 0.7 mm. As shown in FIG. 15, the bowl portion 30 has an outer diameter of about 80 mm and the recess 34 is defined as a toroidal cavity defined by tapering sidewalls, which cause the recess 34 to taper from an inner diameter of about 40 mm at the bottom of the recess 34 to an outer diameter of about 65 mm near the top of the recess 34. The upwardly extending feature 48 has a semi-conical shape defined by sides that taper so that the bottom diameter of the upwardly extending feature 48 is about 25 mm and the top diameter (circumference of the relatively flat surface 49) is about 16 mm. The top of the air duct 58 has a diameter of about 10 mm.

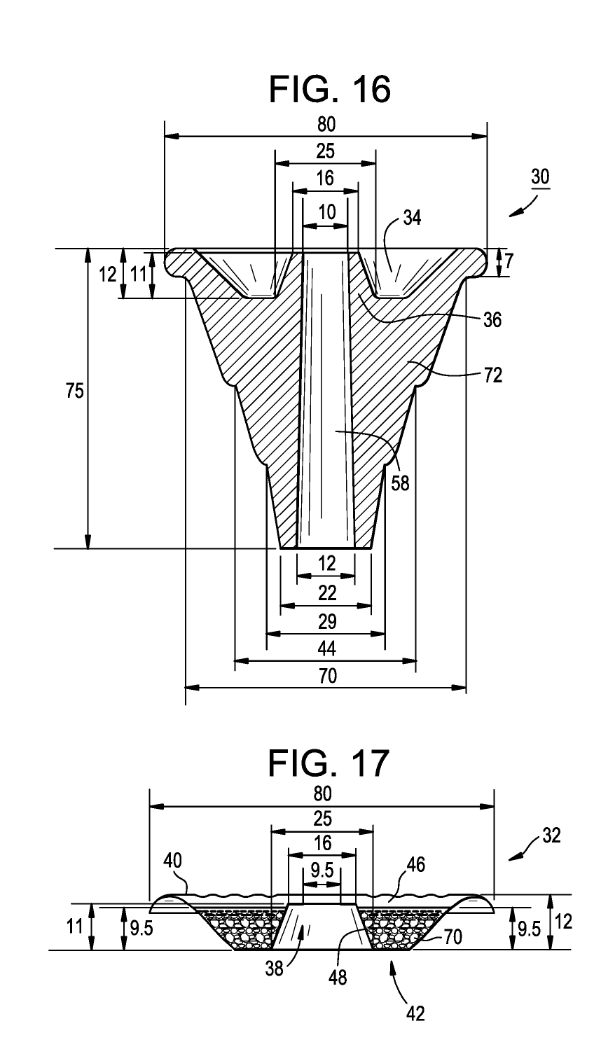

FIG. 16 shows exemplary cross-sectional dimensions of the bowl portion 30. A body 72 of the bowl portion 30 may be a smooth conical shape, or have successively widening cone sections, having diameters that successively increase. For example, the body 72 of the bowl portion 30 has successive outer diameters of 22 mm, 29 mm, 44 mm and 70 mm.

The body 72 defines the recess 34, which has an overall depth of about 12 mm from a bottom surface of the recess 34. The recess 34 is also defined by the upwardly extending feature 48, which has a height as measured from the bottom portion to the relatively flat surface 49 of about 11 mm.

The lower component 40 of the cartridge 32, as shown in FIG. 17, has a shape and dimensions that correspond to the shape and dimensions of the recess 34 of the bowl portion 30, so that the cartridge 32 is restricted from lateral movement (i.e., movement having a directional component perpendicular to a longitudinal axis of the air duct 58). In this example, the receiving portion 38 has a height (from the bottom surface of the cartridge 32 to the hole 50) corresponding to the height of the upwardly extending feature 48 of the bowl portion 30, i.e., about 11 mm. The gap 56 in this example has a thickness of about 1 mm.

Advantages of embodiments described herein include the provision of a disposable cartridge, which avoids the need to have a user add his or her own fluid. In addition, the cartridge can be adapted for existing shisha bowls or other smoking devices, such that need for new equipment is reduced or minimized, and also preserves the look and feel of a traditional hookah or shisha smoking device.

Another advantage is that the shisha bowl assembly 18 provides a potentially healthier alternative, as the cartridges contain the materials that simulate the original shisha experience of producing smoke and flavor, yet without the use of tobacco burning. The process of smoke production is substituted with vapor which is the by-product of heating glycerin. The shisha bowl assembly described herein can employ materials approved for use by appropriate regulatory bodies (e.g., the U.S. Food and Drug Administration) as an alternative to tobacco products. In addition, the vaporizing fluid may not incorporate any nicotine if desired, thereby further improving the potential health effects.

Many modifications of the embodiments described herein as well as other embodiments may be evident to a person skilled in the art having the benefit of the teachings presented in the foregoing description and associated drawings. It is understood that these modifications and additional embodiments are captured within the scope of the contemplated invention which is not to be limited to the specific embodiment disclosed.

What is claimed is:

1. A vaporizing device comprising:
a removable cartridge defining an internal cavity and configured to be disposed on a vapor smoking device; and
a plurality of porous granules disposed within the internal cavity, the plurality of porous granules having a vaporizing liquid absorbed into the plurality of porous granules.

2. The vaporizing device of claim 1, further including a bowl portion having a recess configured to receive a lower component of the removable cartridge.

3. The vaporizing device of claim 2, wherein the bowl portion is configured to be operably connected to the vapor smoking device.

4. The vaporizing device of claim 2, wherein the vapor smoking device is a shisha device and the bowl portion is configured to be operably connected to the shisha device.

5. The vaporizing device of claim 2, wherein:
the bowl portion defines a socket protruding upwardly through the recess and configured to engage a similarly shaped receiving portion defined in the removable cartridge.

6. The vaporizing device of claim 2, wherein:
the removable cartridge is fabricated from aluminum; and
the bowl portion is fabricated from clay.

7. The vaporizing device of claim 1, wherein the removable cartridge is fabricated from aluminum.

8. The vaporizing device of claim 1, wherein the removable cartridge further includes:
an upper component and a lower component, which when assembled form the internal cavity;
the upper component includes one or more holes disposed through a surface thereof and arranged in a plurality of concentric circles surrounding a central point of the surface, and one or more heat distribution grooves disposed on the surface and between a pair of corresponding concentric circles;
the lower component includes a receiving portion formed by an upwardly extending feature terminating at a relatively flat surface, the upwardly extending feature having a conical shape, the relatively flat surface include a hole disposed therethrough and through the lower component.

9. A bowl assembly for a vapor smoking device, the bowl assembly comprising:
a removable cartridge defining an internal cavity and configured to be disposed on a vapor smoking device;
a plurality of porous granules disposed within the internal cavity, the plurality of porous granules having a vaporizing liquid absorbed into the plurality of porous granules; and
a bowl portion having a recess configured to receive a lower portion of the removable cartridge.

10. The bowl assembly for a vapor smoking device of claim 9, wherein the bowl portion is configured to be operably connected to a shisha device.

11. The bowl assembly for a vapor smoking device of claim 9, wherein:
the bowl portion defines a socket protruding upwardly through the recess and configured to engage a similarly shaped receiving portion defined in the the removable cartridge.

12. The bowl assembly for a vapor smoking device of claim 9, the bowl assembly further comprises:
a heating element disposed therein.

13. The bowl assembly for a vapor smoking device of claim 9, wherein:
a heating element is disposed on the vapor smoking device in proximity to the bowl assembly.

14. The bowl assembly for a vapor smoking device of claim 9, wherein the removable cartridge is fabricated from aluminum.

15. The bowl assembly for a vapor smoking device of claim 14, wherein:
the bowl portion is fabricated from clay.

16. The bowl assembly for a vapor smoking device of claim 9, wherein the removable cartridge further includes:
an upper component and a lower component, which when assembled form the internal cavity;
the upper component includes one or more holes disposed through a surface thereof and arranged in a plurality of concentric circles surrounding a central point of the surface, and one or more heat distribution grooves disposed on the surface and between a pair of corresponding concentric circles;
the lower component includes a receiving portion formed by an upwardly extending feature terminating at a relatively flat surface, the upwardly extending feature having a conical shape, the relatively flat surface include a hole disposed therethrough and through the lower component.
